(19) United States
(12) Patent Application Publication
(10) Pub. No.: US 2025/0248761 A1
(43) Pub. Date: Aug. 7, 2025

(54) INCORPORATING VESSEL SEALING FUNCTIONALITY INTO SURGICAL LASER INSTRUMENTS

(71) Applicant: Max Shurgalin, Bedford, MA (US)

(72) Inventor: Max Shurgalin, Bedford, MA (US)

(73) Assignee: Healthilase Surgical, Inc., Bedford, MA (US)

(21) Appl. No.: 18/856,541

(22) PCT Filed: Apr. 10, 2023
(86) PCT No.: PCT/US2023/018095

§ 371 Date: Oct. 11, 2024

(51) Int. Cl. A61B 18/28; A61B 17/29; A61B 18/00; A61B 18/08; A61B 18/14; A61B 18/22

(52) U.S. Cl. CPC A61B 18/28; A61B 18/085; A61B 18/1445; A61B 2017/2929; A61B 2017/2944; A61B 2018/00577; A61B 2018/00601; A61B 2018/0063; A61B 2018/00982; A61B 2018/2272

(57) ABSTRACT

Surgical laser instruments with vessel sealing functionality and method of incorporating such functionality into laser instruments are disclosed. Vessel sealing functionality built into instruments for laser surgery involves a hollow mechanical support component, a laser beam delivery conduit disposed in it and an effecter for grasping, compressing, heating and fusing tissue attached to distal end of the hollow mechanical support component. The effecter includes jaws with at least one energy transfer element for conveying energy to tissue. The effecter is actuatable between open state for receiving tissue, closed state for compressing and fusing tissue and a retracted state for using surgical laser beam. The distal segment of the instrument may be actuatable within a range of angles and also rotationally actuatable. In some embodiments electrical energy is utilized for fusing tissue and in other embodiments the surgical laser beam is used as a source of energy for tissue fusing.

### TECHNICAL FIELD

The present invention generally relates to laser surgery instruments, and specifically to techniques and devices that incorporate vessel sealing functionality into surgical laser instruments without compromising capacity for precise incising and ablating of tissue with surgical laser beam.

### BACKGROUND

Lasers became well-established instrument in surgical practice, often cited as preferred tool for precision surgery and microsurgery, where surgical accuracy enabling most retention of healthy tissues and organ function is of highest benefit to a patient. One critical consideration in performing precision laser surgery is surgeon's ability to accurately aim the laser beam to target tissue, supported by laser beam delivery and manipulation instruments, and therefore such surgical laser instruments are designed to offer excellent tissue target spot visualization and beam aiming accuracy. Another critical consideration in performing precision laser procedures, as in any surgery, is minimization of bleeding caused by cutting through blood vessels and capillaries in order to prevent adverse physiologic effects associated with blood loss. Control of bleeding during surgery, meaning stoppage or reduction of bleeding, is termed surgical haemostasis. In many surgical procedures, cutting through not only small capillaries but through larger blood vessels, and in general through other bodily vessels, ducts or tubular organs, is also needed thus requiring vessel ligation and vessel sealing. Vessel ligation or sealing is achieved by applying mechanical pressure and closing the vessel lumen in required location and then applying some form of energy to heat up the compressed vessel tissue and fuse or “weld” it by thermally induced tissue coaptation process. Bipolar electrosurgical or ultrasound energy instruments are often used for vessel sealing during surgery, for example, LigaSure™ device from Medtronic corporation. Such vessel-sealing devices have to be interchanged with laser cutting and ablating devices during surgical procedures involving laser. In small and tight surgical spaces and in minimally invasive procedures two separate instruments cannot be kept both at the same time and therefore critical time is lost doing device exchanges and also more bleeding can occur. Clearly a need exists in surgical practice to combine precision incising and ablating capability afforded by a surgical laser beam with vessel sealing functionality all in one instrument, thus reducing blood loss between instrument changes as well as removing a burden of instrument changes during surgery every time when a vessel has to be ligated. Desired are methods and devices for incorporating into surgical laser instruments functionality for vessel sealing, which can be invoked when needed and which does not interfere with performing precise surgical incising and ablating of tissue with the laser beam when there is no vessel or other tubular organ to ligate.

Most devices for vessel sealing have necessarily a clamping mechanism such as pivotably coupled jaws and means for applying heat to tissue compressed between the jaws, which in most cases involves electrical energy, for example RF energy, or ultrasound energy. In case of bipolar RF devices, a mechanical knife to cut through fused tissue is often utilized and in ultrasonic devices one jaw is also an ultrasonic scalpel. Some devices feature direct conductive heating of tissue clamped between the jaws by means of electric heaters embedded into the jaws, for example, Altrus™ device from ConMed corporation. Various designs and features for energy-based vessel sealers are found in many issued patents and patent applications. For instance, the patent application US 2002/0188294A1 describes endoscopic forceps for sealing and dividing tissue and includes details of jaw mechanism, means for controlling jaw action and how a user operates the device. The heat is applied to tissue in that device by RF energy in bipolar manner, that is, with electric current flowing from one jaw to another through tissue clamped between the jaws and heating up the tissue.

The relative simplicity of applying RF energy and ultrasonic energy to heat tissue compressed between two pivoting jaws has led to proliferation of such devices on the market compared to devices which use other energies such as optical energy, that is, the energy carried by light. Using optical energy for vessel sealing may have certain advantages compared to electrical or ultrasound energy such as more precise highly localized application of energy to tissue. One device for vessel sealing using clamp or jaws for applying pressure to tissue mechanically and then optical energy to apply heat for tissue fusion was disclosed in the patent U.S. Pat. No. 5,336,221. The device has a clamp with one jaw having a concave surface for engaging with tissue and designed to tightly close off vessel lumen. A plurality of optical fibers is used to deliver laser energy to tissue and distribute it to achieve proper heating of clamped tissue. The optical radiation is used only in conjunction with a clamp and there is no any mechanism allowing use of laser beam for tissue incising and ablating.

Similarly, the patent U.S. Pat. No. 9,402,685B2 describes vessel-sealing device that utilizes optical energy for tissue fusion process. The instrument has pivoting jaws for compressing vessel tissue, one jaw having light distribution element and another jaw having spectrally selective light-reflecting material. The design enables illumination of compressed tissue with light that is absorbed by the tissue causing it to heat up. The light is delivered to the light distribution element from a light source by a light guide element and the light source can be a laser. The design also features a cutting member for cutting tissue, which also functions to deliver light to tissue to cause coagulation while cutting. The patent U.S. Pat. No. 10,925,662B2 describes a similar design with pivoting jaw members, which form a cavity when closed thus compressing vessel tissue around cavity and fusing vessel tissue trapped in the jaw's cavity. Another related approach was presented in the patents U.S. Pat. Nos. 10,925,670B2 and 10,893,908B2, where optical energy for heating compressed tissue is delivered into tissue by mechanism of frustrated total internal reflection causing light to escape optical confinement within the jaws upon contact with tissue and heat the compressed tissue between the jaws. In all cases, optical energy is only used for heating up tissue between the jaws and all described instruments have no any laser beam for precision tissue cutting and ablation.

A laparoscopic articulating handpiece for waveguides was disclosed in the patent U.S. Pat. No. 10,426,546B2. That instrument is designed specifically for precision laser surgery. Surgical laser radiation is delivered via optical fiber or waveguide inserted into the instrument and the laser beam out of the optical fiber or waveguide is used to perform laser surgery. The design of the handpiece allows articulation of the distal end of the instrument and good aiming of the laser beam coming out of the waveguide or fiber held in the instrument. Additional functionality for tissue manipulation and for grasping tissue is enabled by adding distal end features such as cutting and grasping jaws, which feature a distal orifice or port through which the laser beam is directed when jaws are opened. However, such jaws limit surgeon's ability to reach into smaller and constrained surgical spaces and they block surgeon's view for precise laser beam aiming. The device described in the patent U.S. Pat. No. 10,426,546B2 is a surgical laser instrument with additional features but lacking functionality for vessel ligation.

It must be emphasized that the benefits of laser surgery are realized when incising and ablating of tissue is performed with a very well controlled small focused laser beam precisely aimed at the target tissue spot while surrounding living tissue and critical structures remain unaffected and healthy. Safeguarding healthy tissues and critical structures is the purpose and major advantage of precision laser surgery. In that regard, addition of vessel sealing functionality must not interfere with precise and well controlled delivery of the surgical laser beam, especially so in microsurgery procedures in the medical fields of otolaryngology and reproductive surgery. A surgeon needs a laser surgery tool providing the capability to perform vessel ligation without changing instrument in hand and that is not affecting his or her ability to perform laser incising and ablating of tissue with precision. None of the previously disclosed devices appears to be addressing the need for such tool. It is the objective of this invention to fulfil that need.

### SUMMARY OF INVENTION

The present invention features an instrument for precision laser surgeries with build-in functionality for vessel ligation and sealing.

In one aspect of the present invention, the instrument has an outer cannula and a handle and allows a surgeon to perform minimally invasive surgical procedures, for example, laparoscopic surgeries.

In another aspect of the present invention, the instrument has a movable small profile inner cannula for delivery and accurately aiming of surgical laser beam, enabling excellent tissue target spot visualization and control of surgical laser beam application, and allows a surgeon to perform laser surgical procedures with precision. Delivery of surgical laser beam is generally done by an optical fiber or waveguide inserted and secured in the inner cannula.

Yet in another aspect of the present invention, the instrument has a vessel sealing effecter featuring jaw members equipped with means for heating and fusing tissue grasped and compressed by the jaw members, and allows a surgeon to perform vessel ligation without changing instrument in hand. The vessel sealing effecter is actuatable between three states, an open state for receiving tissue, a closed state for compressing and fusing tissue, and a retracted state. In the retracted state, the effecter jaw members are placed into a compact arrangement while the inner cannula is moved forward beyond the vessel sealing effecter thus providing a surgeon with means of accurate laser beam aiming for incising and ablating tissue. In the closed state, a surgeon can adjust compression of tissue by the effecter jaw members.

Yet in another aspect of the present invention, the instrument's distal section, which includes the movable inner distal portion of the inner cannula and the vessel sealing effecter, is actuatable within a range of angles with respect to the main length of the outer cannula at least in one bending plane, enabled by including into the outer and the inner cannulas bendable and actuatable portions. In addition, the outer cannula section that is attached to the instrument handle is rotationally actuatable to allow rotating the bending plane of the distal section with respect to the device handle. Rotating the cannula and actuating the distal section of the instrument to point in different directions gives a surgeon ability to exercise much greater control over how target tissue is visualized, how surgical laser beam is aimed, and how a vessel is grasped and sealed.

Yet in another aspect of the present invention, the means for heating and fusing tissue, also referred to as the energy transfer elements, include RF electrodes and sensors disposed in the effecter jaw members. In other embodiments, the energy transfer elements include electric heaters and sensors disposed in the effecter jaw members. Some embodiments feature the energy transfer elements that include surgical laser beam absorptive elements, or surgical laser beam reflective elements including scattering reflectors, or media transparent to surgical laser beam, or combinations of all those, as well as sensors, disposed in the effecter jaw members. In those embodiments, surgical laser beam is utilized as a source of optical energy for heating and fusing tissue, and a pass-through opening for the surgical laser beam is available when the effecter is in its closed state thus allowing the laser beam to reach the energy transfer elements in the effecter jaw members.

The present invention also features a method of incorporating vessel sealing functionality into surgical instruments for laser surgery by utilizing a movable laser beam delivery conduit placed inside an outer hollow component extended from a handle, and attaching at the end of the outer hollow component a means of grasping, compressing, heating and fusing tissue, which includes jaws and actuated between three different states by cooperative action of jaws actuation controller mechanism and moving the movable laser beam delivery conduit. The three different states are closed state, when the jaws are closed in front of the movable laser beam delivery conduit for holding and fusing tissue, open state, when the jaws are open in front of the movable laser beam delivery conduit and ready to grasp tissue, and retracted state, when the movable laser beam delivery conduit is passed between the jaws and the jaws are in a compact arrangement alongside the movable laser beam delivery conduit.

All the above aspects and further details of the present invention are apparent in the following detailed description of the invention and the accompanying figures.

### BRIEF DESCRIPTION OF DRAWINGS

FIG. 1 provides a general illustration of the present invention.

FIG. 2 presents further details of the present invention.

FIG. 3A and FIG. 3B show an embodiment of the vessel sealing effecter and the effecter controller.

FIG. 4 is an illustration of the vessel sealing effecter in its retracted state.

FIG. 5A and FIG. 5B provide an illustration of the vessel sealing effecter being actuated via cooperative action of the effecter controller and motion of the inner distal portion and transitioning from the retracted state into the open state.

FIG. 6 is an illustration of the vessel sealing effecter in its open state.

FIG. 7 is an illustration of the vessel sealing effecter in its closed state.

FIG. 8 provides an illustration of the vessel sealing effecter in its closed state compressing tissue and shows basic principles of regulating compression of tissue and using surgical laser beam as a source of energy for heating and fusing tissue.

FIG. 9 shows exemplary mechanisms in the handle for regulating tension of the effecter controller cables and for moving the inner cannula.

FIG. 10 shows an embodiment of energy transfer element with electric heater and temperature sensor.

FIG. 11A and FIG. 11B show an embodiment of the energy transfer element with surgical laser beam absorptive element and reflectors and an example of absorption profile for the laser beam absorptive element, respectively.

FIG. 12A and FIG. 12B show an embodiment of the energy transfer element with surgical laser beam scattering reflector, surgical laser beam absorptive element and transparent medium and illustrate schematically use of optical energy delivered by surgical laser beam.

### DESCRIPTION OF INVENTION

The readers of this disclosure skilled in the art should recognize that the term vessel as used herein refers in general to any bodily vessel, duct or tract and not just blood vessel. Terms vessel ligation and vessel sealing are meant to describe the same effects in tissue and resulting in the same outcome, which is complete closure of the vessel and the vessel tissue fused. Furthermore, the particulars presented in the drawings and the detailed discussion below are by way of example and are for purposes of only illustrating preferred embodiment towards the goal of clearly elucidating the principles and conceptual aspects of the present invention. In that regard, it is to be understood that the present invention is not limited to the details of construction and arrangement of components as set forth in the following description and in the drawings, and the terminology employed should not be regarded as limiting either. Skilled in the art can practice many variations without departing from the fundamentals of the present invention.

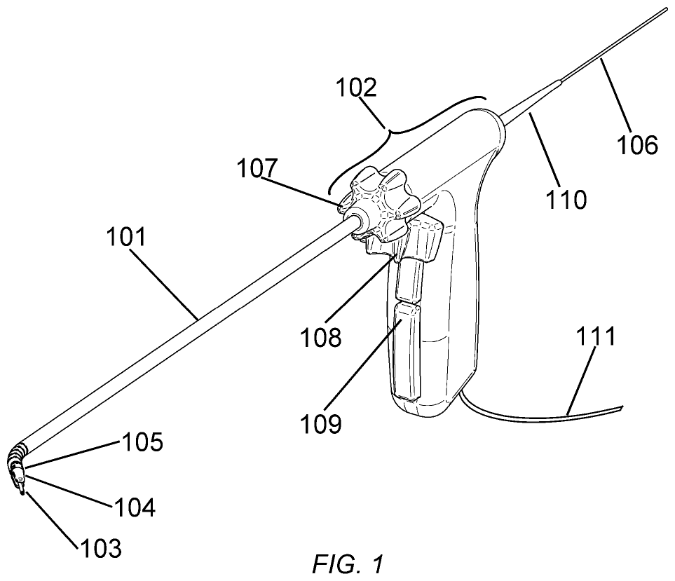

FIG. 1 shows a generic instrument utilizing an embodiment of the present invention. An outer cannula 101 is attached to a handle 102 and an inner cannula 103 is disposed inside the outer cannula 101, which serves as mechanical support framework. At the distal end of the outer cannula 101, an effecter 104 for grasping, compressing, heating and fusing tissue, that is, the vessel sealing effecter, is integrated with an effecter controller 105. The term distal in the context of this specification has a meaning of being further away from the instrument handle and closer to tissue to be treated, while the term proximal has a meaning of being closer to the instrument handle. In these terms, the outer cannula 101 has outer distal and outer proximal portions, which are connected together by a bendable outer connection portion to form the whole outer cannula 101 as shown in FIG. 1, thus making the outer distal portion actuatable within a range of angles and allowing the outer distal portion to be positioned at different angles with respect to the outer proximal portion. The range of angles typically is from 0 degrees to 90 degrees for each side. Skilled in the art are familiar with how bendable and actuatable sections can be designed and how actuation can be enacted, for example, by means of two actuation cables from the handle of the instrument as described in the patent U.S. Pat. No. 10,426,546B2. If the instrument has no bendable and actuatable sections, the outer cannula 101 accordingly is rigid. The inner cannula 103 also has a distal portion and a proximal portion and in instruments with bendable and actuatable sections, the inner cannula 103 or part of it is bendable. That is, the inner cannula 103 is made in part or in whole of a semi-rigid and sufficiently elastic material so that it can bend locally over a range of bending angles under application of bending forces with no kinking, but without any bending forces applied it does maintain its original form. Materials suitable for the bendable inner cannula and the bendable part of the inner cannula include but not limited to semi-rigid and structurally reinforced polymers, for example PEEK and polyimide with stainless steel wire and braid reinforcements, and super-elastic materials, for example nitinol. Making the bendable inner cannula or a portion of it from super-elastic material has an advantage that the inner cannula can act as additional mechanism for controlling the bendable and actuatable sections of the instrument, for example, to facilitate return into straight configuration. In some embodiments the bendable portion of inner cannula is engineered with required properties by structural design combining more rigid elements with more flexible materials and therefore utilizing composite material, for example, incorporating wires made of super-elastic materials such as nitinol into tube made of flexible plastic. It is important to understand that the inner distal portion of the inner cannula 103 does maintain its straight-line shape and does return to its straight-line shape after being forced to bend. The inner cannula 103 is rigid and straight in instruments without bendable and actuatable sections. The inner cannula 103 provides a means for delivery of surgical laser beam with an optical fiber 106 inserted into the inner cannula 103 and secured in it so that the optical fiber end emitting surgical laser beam is located at the distal tip of the inner cannula 103. Therefore, the inner cannula 103 provides a means for aiming surgical laser beam to perform precision incising and ablating of tissue with surgical laser beam emanating from the optical fiber. The diameter of the inner cannula 103, and particularly the diameter of the inner distal portion, is small, not exceeding 2.5 mm in a preferred embodiment. When the instrument is used for incising and ablating tissue, the distal tip of such small profile inner distal portion is positioned forward of the vessel sealing effecter 104 as shown in FIG. 1, thus enabling good tissue target spot visualization and accurate pointing of the surgical laser beam as required for precision laser surgery instruments.

The instrument handle 102 houses mechanisms for different actuations to operate the instrument and accordingly operating controls are located on the handle, for example, a knob 107 for rotating the outer cannula 101, another knob 108 for actuating the distal section and controls 109 for actuating the vessel sealing effecter, which are shown in FIG. 1 only schematically. The handle 102 may also have a strain relief 110 for fiberoptic and electrical connections 111, also shown in FIG. 1 only schematically. The electrical connections 111 are needed in some embodiments in which actuation mechanisms require electrical power and the vessel sealing effecter requires electrical energy such as RF energy. The electrical connections 111 are also needed for electronic signals, including feedback signals for controlling energy supplied to the vessel sealing effecter, signals communicating different states of the vessel sealing effecter, such states explained further in detail below, and sensor signals in embodiments utilizing sensors in the vessel sealer effecter. Some embodiments with electrically powered actuation mechanisms and electrically powered vessel sealer effecter have battery together with embedded electronic controls in the handle and the instrument in that case is battery-powered not requiring electrical cables. Skilled in the art are generally familiar with how to design various mechanical and electrically-powered actuation mechanisms.

Now it is necessary to explain the meaning of cannula and conduit and hollow component terms in the context of this specification. The term cannula and the term conduit and the term hollow component are used to refer to an elongated hollow body, generally of any cross-sectional shape, which on the outside may have the same contour as or different contour than the contour of the hollow, for example, circular inside and rectangular outside, but having discrete axial symmetry about the longitudinal center axis running through the center of and along the length of such hollow body. Cannulas, conduits and hollow components thus defined may have side openings of different shapes along length. Side openings may be oriented certain way to create preferential bending plane in which cannula or conduit or hollow component can bend more easily. Cannulas, conduits and hollow components, and different portions and segments of cannulas, conduits and hollow components, being centered or held centered one to another at a given location generally means that their individual longitudinal center axes, as defined above, coincide at that location. Different portions and segments of cannulas and conduits, such as distal portions or segments, being connected or attached generally means that connection or attachment of a portion and a segment to another element, for example, to another portion or segment, or to a handle, is bendable, rotatable or both bendable and rotatable, or rigidly connected. In the simplest case, a connected or attached portion or segment may simply mean further straight rigid extension of another portion or segment. In an embodiment depicted in FIG. 1, the attachment of the outer cannula 101 to the handle 102 is rotatable and the rotation of the outer cannula 101 and all elements housed inside it is actuated by the knob 107 within 180 degrees in each direction of rotation with respect to the handle 102. Such actuation mechanisms are generally well known to those skilled in the art. One example of a mechanism for rotating cannula or shaft is found in the already mentioned patent application US 2002/0188294A1.

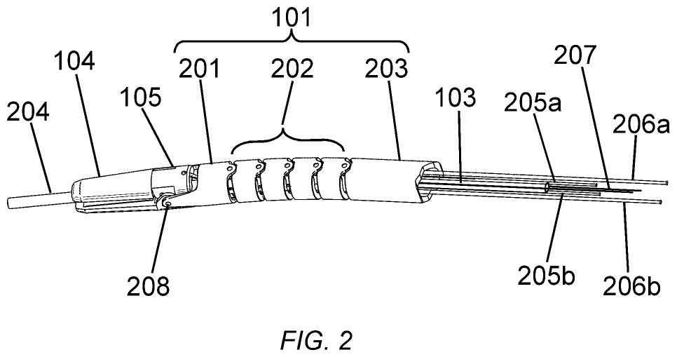

FIG. 2 provides further explanation of the present invention by showing in detail the distal section of the instrument depicted in FIG. 1. The outer cannula 101 comprises an outer distal portion 201, an outer connection portion 202, which in this preferred embodiment are actuatable and bendable, respectively, within a range of angles as already described above, and then an outer proximal portion 203, shown in FIG. 2 in a cutaway manner, revealing the inner cannula 103 inside, the proximal portion of it running to the handle. The inner cannula 103 comprises an inner distal portion 204. The inner distal portion 204 is centered to the outer distal portion 201 and is movable along longitudinal center axis of the outer distal portion 201. The inner distal portion 204 is therefore straight. In this embodiment of the present invention the inner distal portion 204 is movable over such range of distances that upon its movement at least part of it goes through the bendable outer connection portion 202. Therefore, the inner distal portion 204 or part of it, being also a portion of the inner cannula 103, is bendable as already discussed above, and it is engineered and made in part or in whole of appropriate materials so that it maintains its straight shape when not bent and returns to its straight shape after having been bent. Other elements disposed inside the outer cannula 101 around the inner cannula 103 include effecter controller cables 205a and 205b, explained in further detail below, actuation cables 206a and 206b and also electrical cables and wires 207, which are run from the effecter 104 to the handle. The effecter 104 with integrated effecter controller 105 is attached to the outer distal portion 201 by means of two pins 208 on both sides, of which one is visible in FIG. 2.

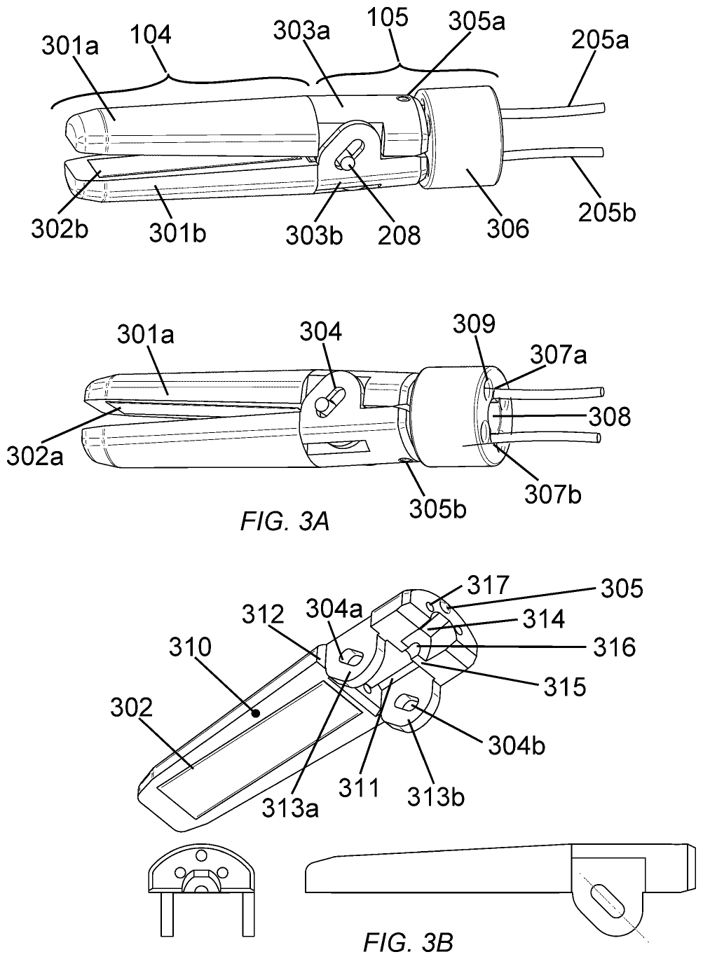

Referring now to FIG. 3A, the effecter 104 with the integrated effecter controller 105 comprises a pair of jaw members 301a and 301b, equipped with energy transfer elements 302a and 302b for conveying energy from an energy source to tissue resulting in heating and fusing of tissue. In some embodiments the energy is optical, supplied by the surgical laser beam. In other embodiments the energy is electrical, such as traditional RF energy, and the energy transfer elements 302a and 302b feature RF electrodes similarly to many existing electrical vessel sealer devices and which skilled in the art are familiar with. In some embodiments the energy is electrical but converted into heat by electric heaters embedded in the energy transfer elements 302a and 302b. The energy transfer elements 302a and 302b include sensors in some embodiments, for example, temperature sensors such as thermocouples and thermistors. In other embodiments the energy transfer elements 302a and 302b include pressure sensors in addition to temperature sensors. The jaw members 301a and 301b are integrated with the effecter controller 105, which is constructed in this embodiment of the following: two jaw actuation portions 303a and 303b, which are rigid extensions of the jaw members and have slotted holes 304, the two pins 208 connecting the jaw actuation portions 303a and 303b on each side of the longitudinal center axis of the outer distal portion utilizing the slotted holes 304, then the effecter controller cables 205a and 205b, which connect to the jaw actuation portions 303a and 303b at locations close to the back and the top and bottom edges shown in FIG. 3A as cable connection points 305a and 305b, and finally a guide 306. The guide 306 directs the effecter control cables 205a and 205b by virtue of having channels 307a and 307b and centers and guides the inner distal portion by virtue of having cannula channel 308. The guide 306 also has pass-through holes 309 for electrical cables and wires. The guide 306 fits into the outer distal portion and is rigidly secured in it. The pins 208 are both aligned with an axis that crosses the longitudinal center axis of the outer distal portion and they are the attachment pins for securing the effecter 104 with integrated effecter controller 105 to the outer distal portion 201 as shown in FIG. 2. The meaning of the pins being aligned with an axis is that the individual center axes of the pins coincide with that axis of pins alignment.

Further understanding of the effecter 104 with the integrated effecter controller 105 is gained from FIG. 3B, which shows a single jaw member with its jaw actuation portion and slotted holes. The slotted holes 304a and 304b have slot angle with respect to the jaw tissue-clamping surface 310 of 45 degrees in this embodiment and in other embodiments the slot angle can be chosen from a range of 30 degrees to 60 degrees. The slotted holes and the slot angle are the features making the jaw members movable both rotationally and translationally about the pins 208. That the jaw members are movable rotationally and translationally allows actuating the effecter 104 between three different states as explained in detail further below. An opening 311 on one side, a recess 312 on the other side and two side extensions 313a and 313b with the slotted holes 304a and 304b are located and dimensioned so that the two identical jaw members with their actuation portions are assembled together as shown in FIG. 3A. The jaw actuation portion has an angled recess 314 approximating the external contour of the inner distal portion. The angled recess 314 also has a chamfered surface as seen in FIG. 3B. A stop ridge 315 is thus formed and its functionality will be apparent from further discussion below. In some embodiments a pass-through opening 316 for surgical laser beam is present, allowing the laser beam to reach the energy transfer elements 302 embedded in the effecter jaw members. In some embodiments pass-through holes 317 for electrical cables and wires are also present, allowing the energy transfer elements 302 to have RF electrodes, electric heaters and sensors.

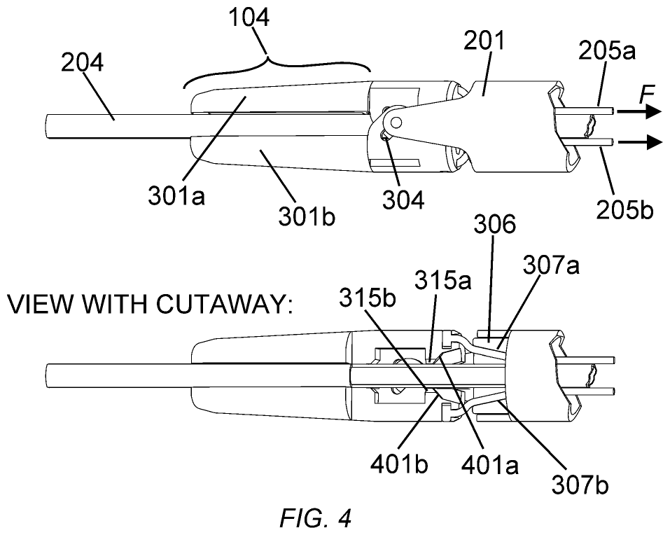

According to the present invention, the effecter 104 is actuatable between a closed state for holding and compressing tissue, an open state for receiving tissue, and a retracted state for allowing visualization of tissue target spot and aiming of surgical laser beam. FIG. 4 depicts the effecter 104 in its retracted state, also showing an additional view with cutaway and explaining the internal details of the effecter 104 and the effecter controller 105. As shown, the inner distal portion 204 is centered by the guide 306 to the outer distal portion 201 and is moved along longitudinal center axis of the outer distal portion 201 to project in distal direction beyond the effecter 104. The maximum distance that the tip of the inner distal portion 204 extends is at least 3 mm and it may be much longer in different embodiments, depending on requirements for surgical laser beam aiming and tissue target spot visualization. Because the inner distal portion 204 is moveable, how much it projects beyond the effecter 104 is adjustable by the user of the instrument up to the maximum distance allowed in a particular embodiment. The jaw members 301a and 301b are closed but separated by the inner distal portion 204 and sitting alongside it so that in the retracted state they remain in compact configuration with outside dimension not exceeding the diameter of the outer cannula. Such configuration is achieved by virtue of the following. The effecter controller cables 205a and 205b guided by the cannels 307a and 307b in the guide 306 are tensioned by forces applied with a mechanism located in the instrument handle and the jaw members 301a and 301b are pulled towards the guide 306 and the outer distal portion 201. Referring to the view with cutaway in FIG. 4, the tip of the inner distal portion 204, being moved in the distal direction, which is to the left in FIG. 4, pushes the jaw members 301a and 301b with their actuation portions away from the outer distal portion 201 by acting upon the angled recesses with chamfered surfaces 401a and 401b and forcing the jaw members 301a and 301b to separate by virtue of the slotted holes 304 allowing not only rotational motion but also translation. Once the tip of the inner distal portion 204 is moved over the stop ridges 315a and 315b, the jaw members 301a and 301b get separated by the maximum separation distance allowed, which is determined by the slot length and slot angle of the slotted holes 304 and which matches the dimension of the inner distal portion 204 in the direction perpendicular to the jaw tissue-clamping surfaces, for example, matches the diameter of the inner distal portion 204 if it is round. At the same time any rotational movement of the jaw members 301a and 301b is blocked because of mechanical constraint imposed by the slotted holes 304 and the stop ridges 315a and 315b sitting against the inner distal portion 204. Once the tip of the inner distal portion 204 passes the stop ridges 315a and 315b, the jaw members 301a and 301b remain in the compact configuration of the retracted state and the inner distal portion 204 is moved further to extend beyond the effecter 104 as shown in FIG. 4. It is now becoming evident that the effecter 104 is actuated between different states by the effecter controller 105 configured to actuate the jaw members 301a and 301b in cooperation with movement of the inner distal portion 204.

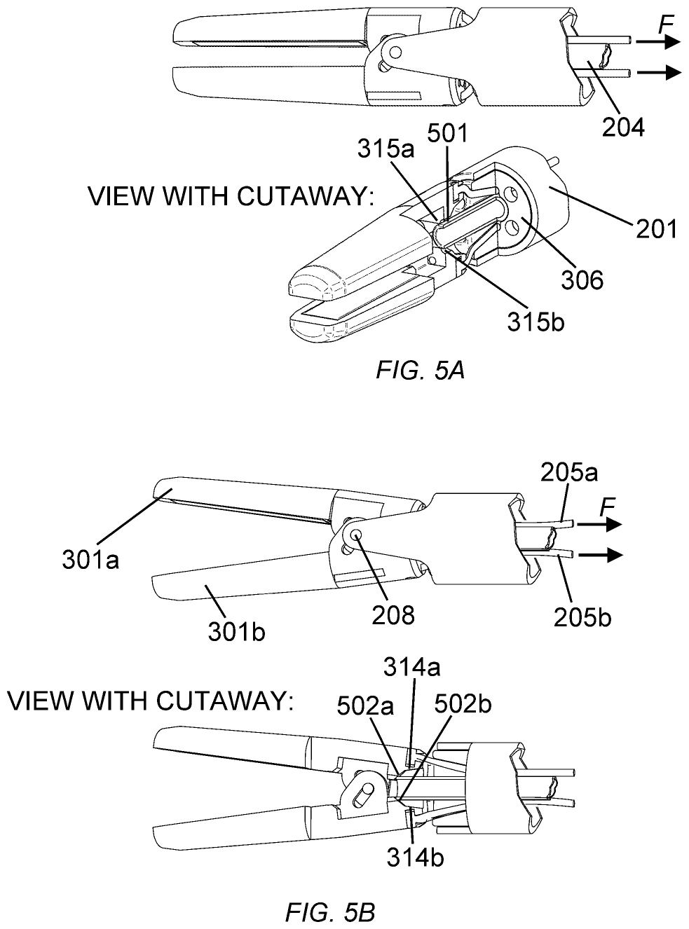

To further elucidate the present invention, FIG. 5A depicts the effecter 104 still in its retracted state but about to be actuated into the open state for receiving tissue, presenting a cutaway view at a different angle. The inner distal portion 204 is moved in the proximal direction, which is to the right in FIG. 5A, and before its tip passes the stop ridges 315a and 315b, the effecter remains in its retracted state. In embodiments where the pass-through opening 316 for surgical laser beam is present as shown in FIG. 3B, the inner distal portion has flats 501 on both sides contacting the stop ridges 315a and 315b, as shown in FIG. 5A in the view with cutaway, so that the laser beam pass-through opening does not interfere with the action of the stop ridges 315a and 315b and provides required mechanical constraint as already described above. Now, referring to FIG. 5B, once the tip of the inner distal portion 204 passes the stop ridges 315a and 315b upon movement in the proximal direction, which is to the right in FIG. 5B, the mechanical constraint is removed and the jaw members 301a and 301b are allowed to pivot about the pins 208 under the pull force of the effecter controller cables 205a and 205b while any translational movement of the jaw members 301a and 301b is prevented by virtue of the inner distal potion 204 pressing against the chamfered surfaces 502a and 502b of the angled recesses 314a and 314b. As the inner distal portion 204 is being moved more in the proximal direction, the chamfered surfaces 502a and 502b of the angled recesses 314a and 314b slide against its tip and the jaw members 301a and 301b rotate more and the effecter is thus transitioning into its open state. Once the tip of the inner distal portion 204 passes the chamfered surfaces 502a and 502b, the jaw members 301a and 301b reach fully open position and the effecter 104 is actuated completely into its open state.

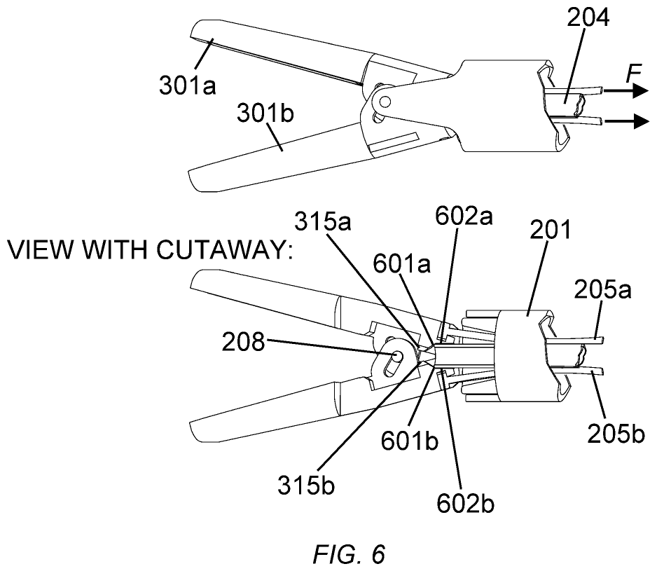

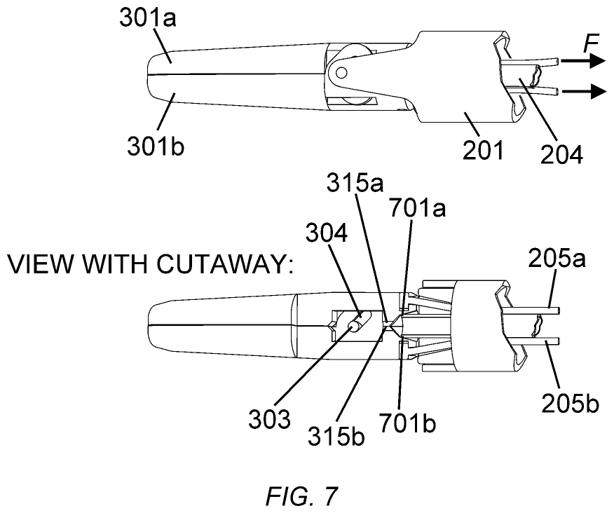

FIG. 6 presents the effecter 104 in its open state for receiving tissue and the view with cutaway shows that the tip of the inner distal portion 204 is located at points 601a and 601b, representing, with reference to FIG. 3B, the edges between the angled recess 314 and its chamfered surface in each of the jaw member actuation portions. The tip of the inner distal portion 204 therefore prevents any translational movement of the jaw members under the pull forces of the effecter controller cables 205a and 205b despite of slotted holes, and the jaw members 301a and 301b remain separated the most under such mechanical constraint. At the same time, the surfaces of the angled recess 314, shown in FIG. 3B, in each of the jaw member actuation portions, shown as 602a and 602b in FIG. 6 in the view with cutaway, rest against the inner distal portion 204 and the jaw members are also prevented from further rotation. As long as the inner distal portion 204 remains in the position depicted in FIG. 6, the effecter stays in the open state. The effecter controller is thus configured to cooperate with motion of the inner distal portion to actuate the effecter between its retracted state and its open state. If the inner distal portion 204 is moved further in the proximal direction, which is to the right in FIG. 6, the mechanical constraint preventing rotational and translational movement of the jaw members about the pins 208 is relaxed and the slotted holes 304 are able to translate along the slot axes. The pull force of the effecter controller cables 205a and 205b makes the jaw members 301a and 301b with their actuation portions to move towards the outer distal portion 201 and to come closer to each other by virtue of slotted holes acting to bring the jaw members together via translation initially and then, once the stop ridges 315a and 315b come against each other, via rotation, about the pins 208. The effecter is then transitioned into the closed state shown in FIG. 7. The effecter controller is thus configured to cooperate with motion of the inner distal portion to actuate the effecter between its open state and its closed state.

In the closed state of the effecter, the jaw members 301a and 301b are pulled with the pull forces of the effecter controller cables 205a and 205b towards the outer distal portion 201 as much as the pins 208 and the slotted holes 304 permit, and brought together by virtue of slotted holes as shown in FIG. 7. The tip of the inner distal portion 204 remains in the position where it is in contact with the chamfered surfaces of the angled recess 314 in each of the jaw member actuation portions, shown in FIG. 7 in the view with cutaway as 701a and 701b. In order to open the jaw members 301a and 301b, the inner distal portion 204 is to be moved in the distal direction, which is to the left in FIG. 7. Upon movement, the tip of the inner distal portion 204 pushes on the chamfered surfaces of the angled recess 314 in each of the jaw member actuation portions and, counteracting the pull forces of the effecter controller cables 205a and 205b, makes the jaw members 301a and 301b to separate from each other via translational and rotational movement allowed by the slotted holes 304 about the pins 208. The jaw members 301a and 301b become fully separated and open and the effecter is thus actuated into its open state. As the inner distal portion 204 is moved in the distal direction more, the jaw members 301a and 301b rotate into closing because of the tip of the inner distal portion acting on the chamfered surfaces of the angled recess 314 in each of the jaw member actuation portions. Once the tip of the inner distal portion 204 reaches the stop ridges 315a and 315b, further movement of the inner distal portion 204 in the distal direction causes the stop ridges 315a and 315b to slide over the inner distal portion 204 thus finishing rotational movement of the jaw members 301a and 301b and actuating the effecter into its retracted state by virtue of mechanical constraint set by the pins 208, the slotted holes 304 and the stop ridges 315a and 315b, as already explained above. The effecter controller is thus configured to cooperate with motion of the inner distal portion to actuate the effecter between its closed state and its open state and then between its open state and its retracted state. It should be noted here that in some embodiments the slotted holes are not straight but curved in order to promote translation of the jaw members as angle between them changes and thus to facilitate actuation of the effecter between its three states, particularly transition from its open state to its closed state.

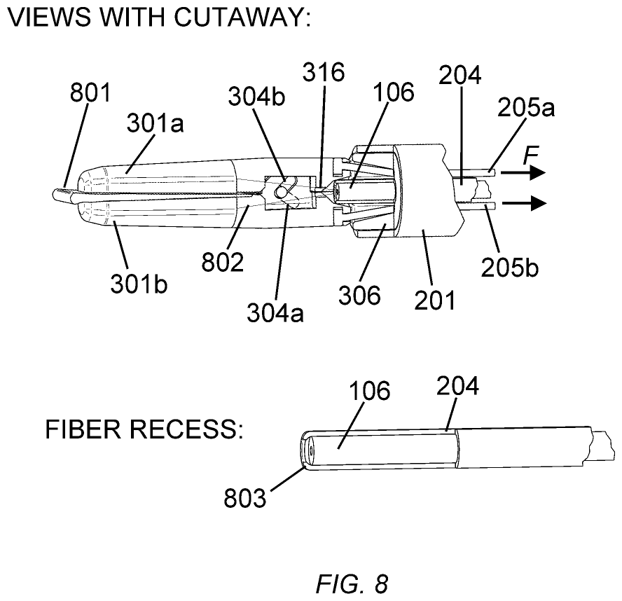

FIG. 8 provides further explanation of the vessel sealing effecter in its closed state, showing tissue 801 compressed between the jaw members 301a and 301b and also in the cutaway showing the optical fiber 106 inside the inner distal portion 204 emitting laser beam 802 through the hole formed by pass-through openings 316, which are shown in FIG. 3B, in each of the jaw actuation portions. The laser beam 802, as shown schematically in FIG. 8, reaches the energy transfer elements embedded in the jaw members 301a and 301b thus providing energy for tissue heating and fusing. The laser beam power is adjusted when used for vessel sealing from power levels used for tissue cutting and ablating, for example, by means of a control signal to the surgical laser when the vessel sealing effecter is closed on tissue. In some embodiments, signals from sensors that are embedded in the energy transfer elements are also sent to the surgical laser to regulate the laser beam power. The optical fiber 106 is secured in the inner distal portion 204 so that the optical fiber end facet is slightly recessed with respect to the tip of the inner distal portion 204 and therefore is protected from damage during mechanical action of the tip of the inner distal portion 204 actuating the effecter as described above. FIG. 8 shows in the additional enlarged view with cutaway such fiber recess in the inner distal portion 204. In some embodiments, a small swage 803 at the tip of the inner distal portion 204 stops the end of the optical fiber 106 and also provides a rounded edge facilitating the described above actuation actions. In other embodiments, the optical fiber may extend beyond the tip of the inner distal portion 204, which has no swage in that case, when the effecter is put into the retracted state and the instrument is used for tissue cutting and ablating with surgical laser beam. The optical fiber is then retracted into the inner distal portion 204 to protect its end facet when the effecter is not in its retracted state and the instrument is used for vessel sealing and such retraction of optical fiber is enacted prior to actuation of the effecter out of its retracted state.

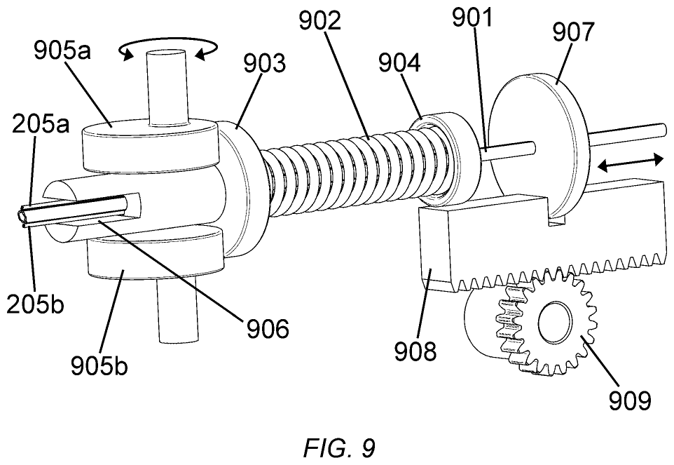

Also shown in FIG. 8 for further clarification are the slotted hole 304b belonging to the actuation portion of the lower jaw member 301b and its counterpart the slotted hole 304a belonging to the actuation portion of the upper jaw member 301a. Because the slotted holes are at an angle to the tissue contacting surfaces of the jaw members 301a and 301b, tissue grasped by the jaw members and forcing them to separate also causes the jaw members to move away from the outer distal portion 201 and the guide 306, counteracting the pull forces of the effecter controller cables 205a and 205b. The magnitude of the pull forces therefore controls how much the tissue is compressed. Adjusting the pull forces of the effecter controller cables 205a and 205b allows regulating tissue compression. Skilled in the art should be familiar with mechanisms to tension cables and to regulate forces applied. One example of such mechanism provided in the instrument handle in some embodiments, which utilizes compression spring and allows regulating tension, is shown in FIG. 9.

FIG. 9 presents exemplary mechanisms located in the handle of the instrument for regulating tension of the effecter controller cables and moving the inner distal portion. The shown exemplary mechanisms also allow rotation of the outer cannula with all components disposed inside it. As already discussed, the inner distal portion is connected to the inner proximal portion that goes into the instrument handle. Therefore, moving the inner distal portion implies moving the inner proximal portion axially. As shown in FIG. 9, the inner proximal portion 901 together with the effecter controller cables 205a and 205b is assembled inside the instrument handle with cable tensioning mechanism comprising compression spring 902, spring holder 903 and washer 904. The spring holder 903 is held concentric with both the outer cannula as per FIG. 1 and the inner proximal portion 901 that is run to the instrument handle inside the outer cannula. The spring holder 903 is allowed rotation to accommodate for rotation of the outer cannula and all components disposed inside it, including the inner proximal portion 901 and the effecter controller cables 205a and 205b, and it is also allowed axial movement by action of tension regulator consisting of two off-center discs 905a and 905b. The effecter controller cables 205a and 205b are attached to the washer 904 and kept tensioned by virtue of the spring 902 being compressed. The inner proximal portion 901 passes through the centers of the spring holder 903 and the washer 904 and is allowed to move freely along axial direction as shown in FIG. 9 by the bi-directional straight arrow. Rotating the off-center discs 905a and 905b synchronously allows adjusting spring compression and regulates tension on the effecter controller cables 205a and 205b and therefore the pull forces exerted on the actuation portions of the jaw members. The spring holder 903 has a slot 906 serving the purpose of rotating the spring holder 903 when the effecter controller cables 205a and 205b and the inner proximal portion 901 rotate in connection with rotation of the outer cannula while allowing translation in response to rotation of the off-center discs 905a and 905b for spring compression adjustment. The inner proximal portion 901 has a disk 907 rigidly attached to it and engaged into a slot of a gear rack 908 driven by a gear 909. The disk 907, the gear rack 908 and the gear 909 constitute an exemplary mechanism for moving the inner distal portion by means of moving the inner proximal portion 901 connected to it. The disk 907 allows rotation of the inner proximal portion 901. The gear 909 in some embodiments is driven by a motor and has an encoder for precise positioning of the gear rack 908 and movement of the inner proximal portion 901 and therefore accurate positioning of the inner distal portion for actuating three states of the effecter. In other embodiments the inner proximal portion 901 has an encoder of translational motion.

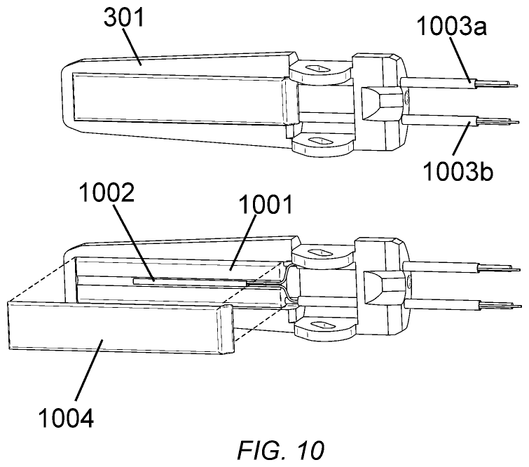

In some embodiments of the present invention the energy transfer elements 302 of the effecter jaw members 301 include RF electrodes or RF electrodes and sensors. In other embodiments the energy transfer elements 302 of the effecter jaw members 301 include electric heaters and sensors. Referring to FIG. 10, the jaw member 301 holds an electric heater 1001 and a temperature sensor 1002, covered by a tissue-contacting membrane 1004. The electric heater and sensor wires are combined into two electrical cables 1003a and 1003b, which are run through the pass-through holes in the actuation portion of the jaw member 301 as shown. The jaw member 301 does not have a pass-through opening for the laser beam in this particular case because the laser beam is not used as a source of energy for tissue fusing. The electrical cables 1003a and 1003b are flexible, allowing movement of the jaw members and actuating the effecter in all its states. The electrical cables 1003a and 1003b are run along the inner cannula as shown in FIG. 2 into the instrument handle where they are flexibly connected to an electronic circuit. In some embodiments more than one heater and more than one temperature sensor are utilized for more refined control of heat distribution and tissue fusing process. In other embodiments pressure sensors are used together with temperature sensors. Skilled in the art should be familiar with using electrical heaters and different sensors in the tissue compressing jaws for tissue fusion and all related electrical and electronic wiring and circuitry as electric vessel sealer devices are commercially available. The membrane 1004 is made of material with good thermal conductivity and having non-stick properties or having non-stick coating on the tissue-contacting surface in order to alleviate the problem of tissue sticking to the jaw members after fusion. In some embodiments the membrane 1004 or the jaw member 301 or both have a pattern of ridges or a tooth-like pattern on the tissue-contacting surfaces to facilitate tissue grasping. The membrane 1004 may also have curvature on its tissue-contacting surface and have flexible area in the middle, allowing transmission of pressure to a pressure sensor underneath it while being rigidly secured in the jaw member 301.

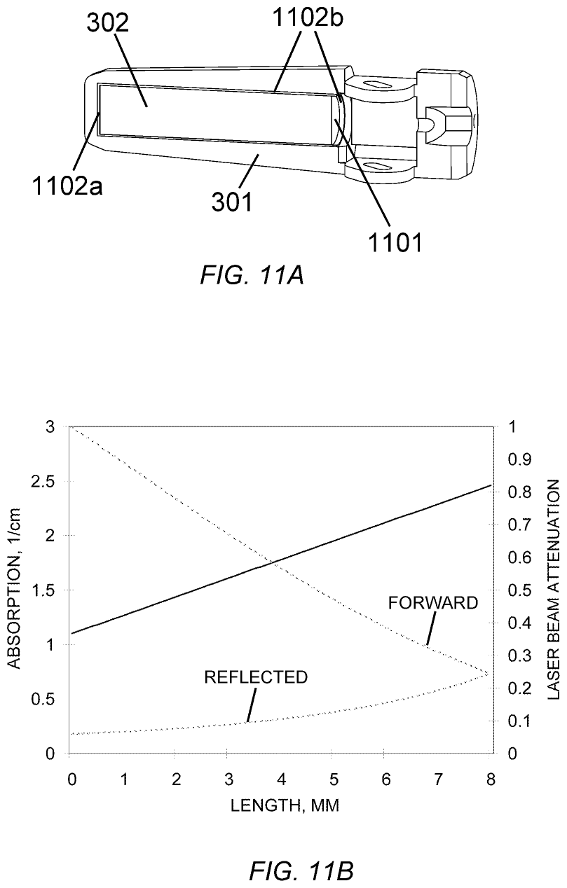

In some embodiments of the present invention the surgical laser beam is utilized as a source of energy for tissue fusing and the energy transfer element 302 in each of the effecter jaw members includes surgical laser beam absorptive elements, which absorb the surgical laser beam, surgical laser beam reflectors, including surgical laser beam scattering reflectors which scatter the surgical laser beam upon reflection, and medium transparent to the surgical laser beam. FIG. 11A presents one such embodiment of the jaw member. The jaw member 301 has the energy transfer element 302 comprising a surgical laser beam absorptive element 1101 and surgical laser beam reflectors 1102a and 1102b. As was already shown in FIG. 8, the surgical laser beam reaches the absorptive element 1101 in each of the jaw members. The absorptive element 1101 absorbs laser light and heats up. The heat flows to tissue compressed between the jaw members and thus in contact with the absorptive elements in each of the jaw members. The absorptive element 1101 has such absorption as to allow the laser beam to propagate through entire length of it. The surgical laser beam reflector 1102a disposed at the end of the absorptive element 1101 reflects back the remaining laser light that is not absorbed in a single pass through the absorptive element. The reflector 1102b disposed around the absorptive element 1101 along its length reflects laser light reaching the boundary of the absorptive element 1101. The absorptive element 1101 together with the reflector 1102b is basically absorptive waveguide for the surgical laser beam with the reflector 1102a at its end. The absorptive element 1101 has absorption profile along its length to maximize absorption of the surgical laser beam in two passes, upon forward propagation and upon reflection back due to the reflector 1102a, and at the same time to make absorption of the surgical laser beam energy and therefore heating of the absorptive element nearly constant along length. FIG. 11B presents an example of required absorption profile for the absorptive element that is 8 mm long. Solid curve in FIG. 11B is absorption coefficient along length and broken curves represent attenuation of the surgical laser beam as it propagates forward and then as it is reflected back, resulting in uniform distribution of absorbed energy with minimal variation along the length of the absorptive element. Helped by thermal conduction, this results in constant heat distribution along the length of the absorptive element. The length of absorptive element is indicated just for example and required absorption profiles can be designed by skilled in the art for different lengths ranging from 2 mm to 20 mm. Examples of materials to construct the surgical laser beam absorptive element include high temperature polymers having intrinsic absorption at the required level at the surgical laser beam wavelength and also doped with absorbent chromophores for absorption at the surgical laser wavelength. The absorption profile shown in FIG. 11B can be achieved in a number of ways, for example, by doping the base material of lower absorption with additional absorbent according to the desired profile. In some embodiments higher absorbing material is used and the desired absorption profile is achieved by reducing absorbent density, for example, by laser micromachining process to create small pores or voids in the bulk of absorbing material. In order to reduce heat loss to the jaw member itself, low thermal conductivity material is used to surround the absorptive element and the surgical laser beam reflectors. In some embodiments the tissue-contacting surface of the absorptive element has non-stick coating to alleviate the problem of tissue sticking after fusion. Some surgical laser energy reaches the tissue directly and some leaks out of the absorptive element via scattering and reflections and reaches the tissue, however because tissue absorbs the surgical laser energy, it helps tissue fusion process. Referring to FIG. 11A, the reflector 1102b surrounding the absorptive element 1101 may also be surgical laser beam scattering reflector to diffusely reflect and scatter part of the surgical laser beam reaching the boundary of the absorptive element back into the absorptive element and thus achieve better uniformity of the surgical laser beam absorption than with specular reflection.

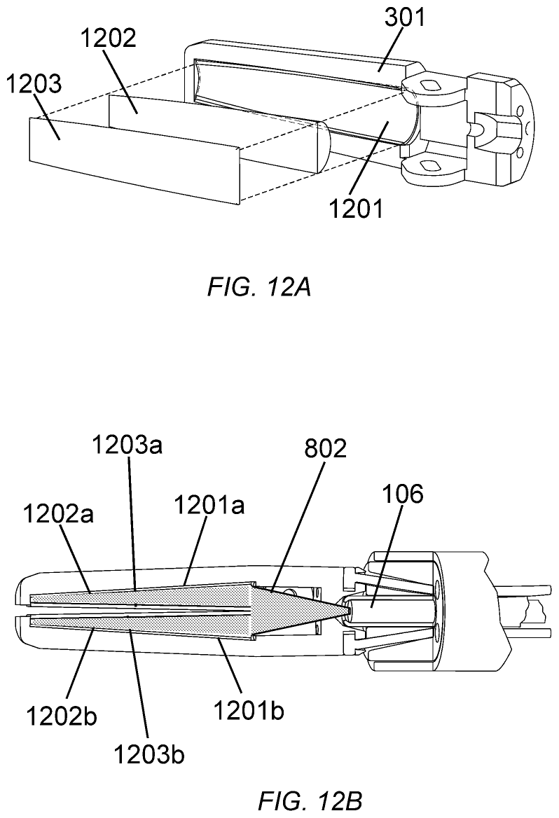

In some embodiments the absorptive element is made in the form of highly absorbent thin plate and the surgical laser beam is directed to it by means of scattering reflector that is designed to reflect and scatter the laser beam along the length of such absorptive element. Referring to FIG. 12A, the jaw member 301 carries surgical laser beam scattering reflector 1201, transparent medium 1202, which is transparent to the surgical laser beam at its wavelength and allows the laser beam propagation along the length of the scattering reflector 1201, and the absorptive element 1203, which may have non-stick coating on its tissue contacting surface. As shown schematically in the cutaway view of the jaw members with the energy transfer elements in FIG. 12B, the surgical laser beam 802 out of the optical fiber 106, while propagating through transparent media elements 1202a and 1202b, is reflected and scattered onto the absorptive elements 1203a and 1203b in both jaw members by the surgical laser beam scattering reflectors 1201a and 1201b. The absorptive elements 1203a and 1203b heat up and transfer heat onto tissue compressed between the jaw members. Skilled in the art should in general be familiar with how to design scattering reflectors and position them with respect to the laser beam. For example, some scattering reflectors for directing laser beam out of an optical fiber directly onto tissue are described in the patent U.S. Pat. No. 10,813,695B2.

The above embodiments illustrated by the referenced figures portray essential principles and conceptual aspects of the present invention, teaching surgical instruments for laser surgery with built-in vessel sealing functionality. From the above descriptions it is also evident that for the most part incorporating vessel sealing functionality into surgical instruments for laser surgery is a method of the following steps. First, providing a hollow mechanical support component with an outer distal segment and an outer proximal segment extending from an instrument handle, which are connected by means of an outer connection segment. Then placing a laser beam delivery conduit inside the hollow mechanical support component. The laser beam delivery conduit includes a conduit distal portion, which is held centered to the outer distal segment of the hollow mechanical support component. Then making the laser beam delivery conduit movable in such manner that the conduit distal portion is movable, preferably along longitudinal center axis of the outer distal segment with respect to the distal end of the outer distal segment, which is the distal end of the instrument. Then attaching a means of grasping, compressing, heating and fusing tissue to the outer distal segment at the distal end, such means comprised of a set of jaws, and equipping such jaws with energy transfer elements for conveying optical energy or electrical energy including RF energy or both to tissue for heating and fusing it. Then assembling the jaws with a controller mechanism for actuating the jaws, such controller mechanism arranged to cooperate with motion of the conduit distal portion to actuate the jaws between a closed configuration, when the jaws are closed in front of the conduit distal portion for holding and fusing tissue, an open configuration, when the jaws are open in front of the conduit distal portion for receiving tissue, and a retracted configuration, when the conduit distal portion is moved between the jaws and leaving the jaws alongside the conduit distal portion. Finally, providing mechanisms in the instrument handle to move the laser beam delivery conduit and to operate the jaws controller mechanism. Additional steps may include using pins aligned with axis crossing the longitudinal center axis of the outer distal segment and making the jaws movable rotationally and translationally about such pins and making the outer distal segment actuatable within a range of angles. The step of equipping the jaws with energy transfer elements may include assembly of the energy transfer elements with RF electrodes or with electric heaters, or with a combination of surgical laser beam absorptive elements, medium transparent to surgical laser beam and surgical laser beam reflectors including scattering reflectors, such combination including all or some of the elements in single or multiple quantities, and also may include assembly of the energy transfer elements with sensors, for example, temperature sensors.

The above descriptions and figures give a number of exemplifications demonstrating how to practice the present invention. It is to be understood however that the present invention is not limited to the exact embodiments and methods described above, various modifications may be considered therein by those skilled in the art without departing from essential principles and the scope of the present invention as defined in the appended claims.

1. A surgical instrument, comprising:
an outer cannula for providing mechanical support framework, wherein said outer cannula comprises an outer distal portion, an outer proximal portion and an outer connection portion, one end of said outer distal portion is an outer distal end, another end of said outer distal portion is connected by means of said outer connection portion to one end of said outer proximal portion and another end of said outer proximal portion is attached to a handle;
an inner cannula for delivery and aiming of surgical laser beam, disposed inside said outer cannula, wherein said inner cannula comprises an inner distal portion centered to said outer distal portion at said outer distal end and movable along longitudinal center axis of said outer distal portion with respect to said outer distal end;
an effecter for grasping, compressing, heating and fusing tissue, disposed at said outer distal end, wherein said effecter is actuatable between a closed state for holding and compressing tissue, an open state for receiving tissue, and a retracted state for allowing visualization of tissue target spot and aiming of surgical laser beam, said effecter comprising jaw members and said jaw members comprising at least one energy transfer element for conveying optical energy or electrical energy or both to tissue; and
an effecter controller, said effecter controller configured to cooperate with movement of said inner distal portion to actuate said effecter between said states.

2. A surgical instrument according to claim 1, further comprising pins aligned with an axis crossing said longitudinal center axis of said outer distal portion, wherein said jaw members are movable rotationally and translationally about said pins.

3. A surgical instrument according to claim 1, wherein said effecter controller is further configured to allow regulating compression exerted by said jaw members on tissue when said effecter is in said closed state.

4. A surgical instrument according to claim 1, wherein said outer connection portion is bendable and said outer distal portion is actuatable within a range of angles.

5. A surgical instrument according to claim 1, wherein said inner cannula or part of said inner cannula is bendable.

6. A surgical instrument according to claim 1, wherein said energy transfer element comprises one or more electric heaters.

7. A surgical instrument according to claim 1, wherein said energy transfer element comprises one or more RF electrodes.

8. A surgical instrument according to claim 1, wherein said energy transfer element comprises one or more sensors.

9. A surgical instrument according to claim 1, wherein said energy transfer element comprises one or more surgical laser beam absorptive elements.

10. A surgical instrument according to claim 1, wherein said energy transfer element comprises one or more surgical laser beam reflectors.

11. A surgical instrument according to claim 1, wherein said energy transfer element comprises medium transparent to surgical laser beam.

12. A surgical instrument according to claim 1, wherein said outer proximal portion is rotationally actuatable.

13. A method of incorporating vessel sealing functionality into surgical instruments for laser surgery, comprising the steps of:
providing a hollow mechanical support component, wherein said hollow mechanical support component comprises an outer distal segment, an outer proximal segment extending from a handle and an outer connection segment connecting said outer proximal segment with said outer distal segment and leaving another end of said outer distal segment to be a distal end;
placing a laser beam delivery conduit inside said hollow mechanical support component, wherein said laser beam delivery conduit comprises a conduit distal portion held centered to said outer distal segment at said distal end;
making said conduit distal portion movable along longitudinal center axis of said outer distal segment with respect to said distal end;
attaching a means of grasping, compressing, heating and fusing tissue to said outer distal segment at said distal end, wherein said means of grasping, compressing, heating and fusing tissue comprises jaws;
equipping said jaws with at least one energy transfer element for conveying optical energy or electrical energy or both to tissue;
assembling said jaws with a controller mechanism for actuating said jaws, wherein said controller mechanism cooperates with motion of said conduit distal portion to actuate said jaws between a closed configuration, when said jaws are closed in front of said conduit distal portion for holding and fusing tissue, an open configuration, when said jaws are open in front of said conduit distal portion for receiving tissue, and a retracted configuration, when said conduit distal portion is moved between said jaws leaving said jaws alongside said conduit distal portion; and
providing mechanisms in said handle for moving said laser beam delivery conduit and for operating said controller mechanism.

14. A method of incorporating vessel sealing functionality into surgical instruments for laser surgery according to claim 13, further comprising the step of using pins aligned with axis crossing said longitudinal center axis of said outer distal segment, wherein said jaws are movable rotationally and translationally about said pins;

15. A method of incorporating vessel sealing functionality into surgical instruments for laser surgery according to claim 13, wherein said energy transfer element comprises one or more electric heaters.

16. A method of incorporating vessel sealing functionality into surgical instruments for laser surgery according to claim 13, wherein said energy transfer element comprises one or more RF electrodes.

17. A method of incorporating vessel sealing functionality into surgical instruments for laser surgery according to claim 13, wherein said energy transfer element comprises one or more sensors.

18. A method of incorporating vessel sealing functionality into surgical instruments for laser surgery according to claim 13, wherein said energy transfer element comprises one or more surgical laser beam absorptive elements and one or more surgical laser beam reflectors.

19. A method of incorporating vessel sealing functionality into surgical instruments for laser surgery according to claim 13, wherein said energy transfer element comprises one or more surgical laser beam absorptive elements, medium transparent to surgical laser beam and one or more surgical laser beam scattering reflectors.

20. A method of incorporating vessel sealing functionality into surgical instruments for laser surgery according to claim 13, further comprising the step of making said outer connection segment bendable and said outer distal segment actuatable within a range of angles.
